(19) United States
(12) Patent Application Publication
(10) Pub. No.: US 2024/0329060 A1
(43) Pub. Date: Oct. 3, 2024

(54) METHOD FOR MEASURING EXTRACELLULAR VESICLES, METHOD FOR ACQUIRING INFORMATION ON NEURODEGENERATION, METHOD FOR ISOLATING EXTRACELLULAR VESICLES, AND REAGENT KIT

(71) Applicant: SYSMEX CORPORATION, Kobe-shi (JP)

(72) Inventors: Igor KUROCHKIN, Kobe-shi (JP); Lausonia RAMASWAMY, Kobe-shi (JP)

(73) Assignee: SYSMEX CORPORATION, Kobe-shi (JP)

(21) Appl. No.: 18/742,611

(22) Filed: Jun. 13, 2024

Related U.S. Application Data
(63) Continuation of application No. PCT/JP2022/039234, filed on Oct. 21, 2022.

(30) Foreign Application Priority Data
Dec. 14, 2021 (JP) 2021-202711

(51) Int. Cl. G01N 33/68; G01N 33/543; G01N 33/58

(52) U.S. Cl. CPC G01N 33/6896; G01N 33/543; G01N 33/58; G01N 2333/33; G01N 2333/4703; G01N 2333/4709; G01N 2333/70596; G01N 2333/916; G01N 2800/28

(57) ABSTRACT

Disclosed is a method for measuring neuron-derived extracellular vesicles in a biological sample in vitro, the method comprising: forming a complex comprising a capture body comprising a tetanus toxin C-terminal fragment, the extracellular vesicle, a detector that specifically binds to a target molecule of the extracellular vesicle, and a labeling substance on a solid phase; and measuring extracellular vesicles having the target molecule based on a signal generated by the labeling substance comprised in the complex.

### CROSS REFERENCE TO RELATED APPLICATIONS

This application claims priority from prior Japanese Patent Application No. 2021-202711, filed on Dec. 14, 2021, entitled “Method for measuring extracellular vesicles, method for acquiring information on neurodegeneration, method for isolating extracellular vesicles, and reagent kit”, the entire contents of which are incorporated herein by reference.

The content of the electronically submitted sequence listing, file name: Q297713_Sequence_listing_as_filed.xml; size: 8,152 bytes; and date of creation: Jun. 11, 2024, filed herewith, is incorporated herein by reference in its entirety.

### FIELD OF THE INVENTION

The present invention relates to a method for measuring extracellular vesicles. The present invention relates to a method for acquiring information on neurodegeneration. The present invention relates to a method for isolating extracellular vesicles. The present invention relates to a reagent kit used for these methods.

### BACKGROUND

An extracellular vesicle (hereinafter, also referred to as “EV”) is a nano-sized (tens of nm to hundreds of nm) membrane vesicle surrounded by a lipid bilayer. An extracellular vesicle is secreted from almost all cells. EVs contain various substances such as proteins, DNA, mRNA, miRNA, lipids, sugar chains, and metabolites derived from cells. In recent years, it has been reported that a substance contained in EV functions as an intercellular information transfer molecule and is involved in various physiological or pathological processes. For example, in the nervous system, EVs containing a causative protein of neurodegenerative disease may be released from cells at the lesion site and spread to other cells, thereby participating in the development of the disease. EVs are present in various biological samples such as blood and urine. For these reasons, it has attracted attention to utilize EV analysis for liquid biopsy.

Typically, ultracentrifugation and size exclusion chromatography (SEC) have been mainly used as a method for collecting EVs from a biological sample. In the ultracentrifugation and SEC, EVs in a biological sample are comprehensively collected. On the other hand, the composition of the contents of EVs varies depending on the cells from which they are derived. For example, it is known that EVs secreted from hippocampal neurons include proteins associated with synaptic vesicles, and EVs secreted from cancer cells include molecules associated with angiogenesis and immune escape. In recent years, as a method for selectively collecting a predetermined EV, an affinity method by capturing a molecule present on the EV surface has been developed. For example, U.S. Pat. No. 9,423,402 describes that EVs in a biological sample were collected using biotinylated CTB and a streptavidin-immobilized particle by utilizing the fact that cholera toxin B (CTB) binds to GM1 ganglioside on the EV surface.

### SUMMARY OF THE INVENTION

The scope of the present invention is defined solely by the appended claims, and is not affected to any degree by the statements within this summary.

An object of the present invention is to provide a means for capturing neuron-derived extracellular vesicles (EVs) (hereinafter, also referred to as “NDEVs”) in a biological sample to enable measurement of NDEVs, a means for evaluating neurodegeneration based on the measurement result of NDEVs, and a means for isolating NDEVs.

The present inventors have completed the present invention by finding that a tetanus toxin C-terminal fragment (hereinafter, also referred to as “TTC”) can selectively capture NDEVs from a biological sample which may contain various EVs.

The present invention provides a method for measuring NDEVs in a biological sample in vitro, the method including: forming a complex containing a capture body containing TTC, NDEV, a detector that specifically binds to a target molecule of NDEV, and a labeling substance on a solid phase; and measuring extracellular vesicles having the target molecule based on a signal generated by the labeling substance contained in the complex.

The present invention provides a method for acquiring information on neurodegeneration of a subject, the method including: forming a complex containing a capture body containing TTC, NDEV, a detector that specifically binds to a target molecule of NDEV, and a labeling substance on a solid phase; and detecting a signal generated by the labeling substance contained in the complex, in which the target molecule is at least one selected from a group consisting of VILIP1, SYT1, UCHL1, SNAP25, GRIA1, GRIA2, Aβ, and phosphorylated tau, the forming and the detecting are performed in vitro, and the measured value obtained in the detecting is as an indicator of neurodegeneration of the subject.

The present invention provides a method for isolating NDEVs in a biological sample in vitro, the method including: binding a capture body containing TTC to NDEV; and removing unreacted free components not bound to the capture body.

The present invention provides a reagent kit for use in the above method, containing a capture body containing TTC.

According to the present invention, it is possible to measure NDEV, evaluate neurodegeneration based on the NDEV measurement result, and isolate NDEV.

### BRIEF DESCRIPTION OF THE DRAWINGS

FIG. 1 is a schematic diagram showing the method for measuring EVs of the present embodiment;

FIG. 2A is a schematic diagram showing an example of the reagent kit of the present embodiment;

FIG. 2B is a schematic diagram showing an example of the reagent kit of the present embodiment;

FIG. 3 is a diagram showing results of Western blot analysis of substances captured by TTC-immobilized particles from cerebrospinal fluid (CSF);

FIG. 4 is a diagram showing results of Western blot analysis of substances captured by TTC-immobilized particles from CSF;

FIG. 5 is a diagram showing results of Western blot analysis of substances captured by TTC-immobilized particles from plasma;

FIG. 6A is a graph showing absorbance of fractions obtained by separating plasma by SEC;

FIG. 6B is a diagram showing results of Western blot analysis of substances in the fractions;

FIG. 6C is a diagram showing results of analyzing substances in fractions containing EVs by enzyme-linked immunosorbent assay (ELISA);

FIG. 7A is a graph showing results of detecting a substance captured by a TTC-immobilized plate from plasma or phosphate buffered saline (PBS) with an anti-CD9 antibody;

FIG. 7B is a graph showing results of detecting a substance captured by a TTC-immobilized plate or a TTC free plate from plasma with an anti-CD9 antibody;

FIG. 7C is a graph showing results of detecting a substance captured by a TTC-immobilized plate from plasma with an anti-CD9 antibody or an isotype control;

FIG. 8A is a graph showing results of detecting a substance captured by a TTC-immobilized plate from plasma or PBS with an anti-GRIA1 antibody;

FIG. 8B is a graph showing results of detecting a substance captured by a TTC-immobilized plate or a TTC free plate from plasma with an anti-GRIA1 antibody;

FIG. 8C is a graph showing results of detecting a substance captured by a TTC-immobilized plate from plasma with an anti-GRIA1 antibody or an isotype control;

FIG. 9A is a graph showing results of detecting a substance captured by a TTC-immobilized plate from plasma or PBS with an anti-SYT1 antibody;

FIG. 9B is a graph showing results of detecting a substance captured by a TTC-immobilized plate or a TTC free plate from plasma with an anti-SYT1 antibody;

FIG. 9C is a graph showing results of detecting a substance captured by a TTC-immobilized plate from plasma with an anti-SYT1 antibody or an isotype control;

FIG. 10A is a graph showing results of detecting a substance captured by a TTC-immobilized plate from plasma or PBS with an anti-UCHL1 antibody;

FIG. 10B is a graph showing results of detecting a substance captured by a TTC-immobilized plate or a TTC free plate from plasma with an anti-UCHL1 antibody;

FIG. 10C is a graph showing results of detecting a substance captured by a TTC-immobilized plate from plasma with an anti-UCHL1 antibody or an isotype control;

FIG. 11A is a graph showing results of detecting a substance captured by a TTC-immobilized plate or a CTB-immobilized plate from plasma or PBS with an anti-CD9 antibody;

FIG. 11B is a graph showing results of detecting a substance captured by a TTC-immobilized plate or a CTB-immobilized plate from plasma or PBS with an anti-CD81 antibody;

FIG. 12A is a graph showing results of detecting a substance captured by a TTC-immobilized plate or a CTB-immobilized plate from plasma or PBS with an anti-UCHL1 antibody;

FIG. 12B is a graph showing results of detecting a substance captured by a TTC-immobilized plate or a CTB-immobilized plate from plasma or PBS with an anti-SNAP25 antibody;

FIG. 12C is a graph showing results of detecting a substance captured by a TTC-immobilized plate or a CTB-immobilized plate from plasma or PBS with an anti-GRIA2 antibody;

FIG. 12D is a graph showing results of detecting a substance captured by a TTC-immobilized plate or a CTB-immobilized plate from plasma or PBS with an anti-SYT1 antibody;

FIG. 12E is a graph showing results of detecting a substance captured by a TTC-immobilized plate or a CTB-immobilized plate from plasma or PBS with an anti-VILIP1 antibody;

FIG. 13A is a graph showing results of detecting a substance captured by a TTC-immobilized plate from plasma of a subject with an anti-VILIP1 antibody; and

FIG. 13B is a graph showing results of detecting a substance captured by a TTC-immobilized plate from plasma of a subject with an anti-SYT1 antibody.

### DETAILED DESCRIPTION OF THE PREFERRED EMBODIMENTS

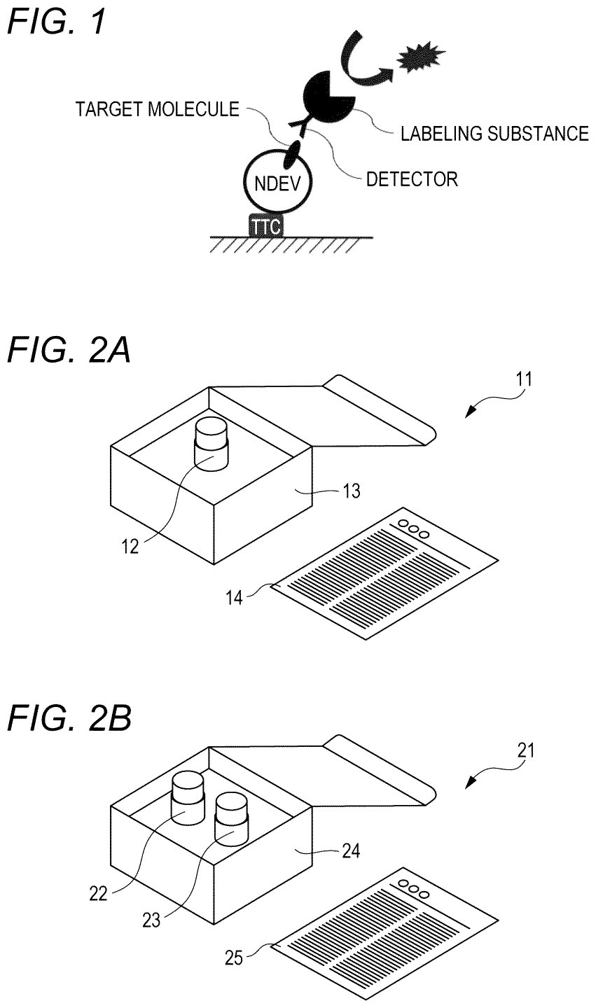

The method for measuring EVs of the present embodiment is a method for measuring NDEVs in a biological sample in vitro, and includes forming a complex described later and detecting a signal generated by the labeling substance contained in the complex. FIG. 1 is a diagram schematically showing an example of a complex containing TTC, NDEV containing a target molecule, a detector, and a labeling substance is formed on a solid phase. In FIG. 1, the labeling substance is an enzyme. By reacting an enzyme with a chemiluminescent substrate, a chemiluminescent signal is generated and can be detected. The type of EV is not particularly limited, and examples thereof include exosomes, ectosomes, microvesicles, microparticles, apoptotic bodies, and the like. The neuron-derived exosome is called NDE. NDEV refers to EV secreted from neurons. The neuron is not particularly limited, and may be either a central nervous system cell or a peripheral nervous system cell. Preferred neuron is a central nervous system cell and includes cranial and spinal neurons.

The biological sample is a sample that may contain NDEV. Since EVs are secreted from cells, examples of the biological sample include a sample collected from a subject, a culture supernatant of a culture containing neurons, and the like. Preferred biological sample is a sample collected from a subject, and examples thereof include blood samples, cerebrospinal fluid (CSF), urine, saliva, tear, lymph fluid, bronchoalveolar lavage fluid, ascites, and the like. The blood sample refers to whole blood, plasma, or serum. If necessary, the biological sample may be diluted with an appropriate aqueous solvent. Examples of the aqueous solvent include water, physiological saline, buffers such as PBS and Tris-HCl, and the like.

Tetanus toxin is initially expressed as a single chain polypeptide of 1315 amino acid residues having a catalytic domain, a translocation domain and a ganglioside-binding domain at the N-terminal side. The polypeptide is cleaved in Clostridium tetani to become an active protein toxin having a structure in which a light chain composed of a catalytic domain and a heavy chain composed of the remaining domains are disulfide bonded. The “tetanus toxin C-terminal fragment” is a C-terminal fragment consisting of amino acid residues at positions 864 to 1315 constituting a ganglioside-binding domain. The method for measuring EVs of the present embodiment utilizes the fact that TTC selectively binds to gangliosides GD1b and GT1b mainly present on the surface of neurons. Since it is considered that GD1b and/or GT1b is also present on the surface of NDEVs, NDEVs in the biological sample can be selectively captured by using a capture body containing TTC.

A base sequence of a gene encoding TTC and an amino acid sequence of TTC are known. TTC itself can be obtained using known DNA recombination technology and other molecular biological techniques. TTC may be a variant in which one or more amino acid residues are deleted, substituted, or inserted in the amino acid sequence of the ganglioside-binding domain of naturally occurring tetanus toxin as long as affinity for ganglioside is not lost. As the amino acid sequence of TTC, for example, a sequence represented by SEQ ID NO: 1 is known. As the amino acid sequence of the variant of TTC, for example, sequences represented by SEQ ID NOs: 2 to 5 are known. These variants have a similar affinity to TTC for ganglioside GT1b. In the present embodiment, a commercially available TTC may be used.

An affinity tag such as a biotin and its analogs, a hapten, or a peptide tag may be added to TTC as long as binding to ganglioside is not hindered. Examples of the biotin and its analogs include biotin and biotin analogs such as desthiobiotin and oxybiotin. Examples of the hapten include 2,4-dinitrophenyl (DNP) group. Examples of the peptide tag include a histidine tag (a peptide consisting of 6 to 10 consecutive histidine residues), FLAG (registered trademark), hemagglutinin (HA), Myc protein, and the like. A method for adding an affinity tag to TTC is known per se. For example, a linker having a biotin group or a DNP group can be used to add a biotin and its analogs or a DNP group to TTC. In addition, TTC fused with a peptide tag can be obtained using known DNA recombination technology and other molecular biological techniques.

The method for measuring EVs of the present embodiment is based on a solid-phase ligand binding assay, and the capture body containing TTC (hereinafter, also simply referred to as “capture body”) is used as a ligand for NDEV. In addition, the detector, the labeling substance, and the solid phase are used. Specifically, NDEV in the biological sample is captured by the capture body, the captured NDEV is immobilized on the solid phase, and the immobilized NDEV is detected by the detector and the labeling substance. The capture body may be TTC itself or TTC to which the affinity tag is added. In the present embodiment, the detector specifically binds to the target molecule of NDEV. The labeling substance is contained in the detector in advance or specifically bound to the detector. That is, in the method for measuring EVs of the present embodiment, first, the complex containing the capture body containing TTC, NDEV, the detector that specifically binds to the target molecule of NDEV, and the labeling substance is formed on the solid phase. Then, a signal generated by the labeling substance contained in the complex is detected.

The target molecule may be a detectable substance of NDEV. The type of the target molecule is not particularly limited, and examples thereof include proteins, nucleic acids, lipids, sugar chains, and combinations thereof. The target molecule may be a substance present on the surface of NDEV or a substance present in NDEV. Preferred target molecule is, for example, a protein commonly present in EVs, a protein expressed in neurons, and the like. Such proteins are known per se.

Examples of the protein commonly present in EVs include CD9, CD63, and CD81, which are members of tetraspanin family. CD9, CD63 and CD81 are known as exosome marker proteins. As shown in Examples described later, CD9, CD63 and CD81 were detected in the substance captured by the capture body containing TTC. This indicates that the capture body containing TTC binds to EV in the biological sample. In the present embodiment, the capture body containing TTC can be used as an EV marker for CD9, CD63, and CD81.

Examples of the protein expressed in neurons include VILIP1 (visinin like protein 1), SYT1 (synaptotagmin 1), UCHL1 (ubiquitin C-terminal hydrolase L1), SNAP25 (synaptosomal-associated protein 25), GRIA1 (glutamate ionotropic receptor 1), GRIA2 (glutamate ionotropic receptor 2), Aβ (Amyloid beta), phosphorylated tau, and the like. VILIP1 is known as one of the visinin/recoverin subfamily of calcium sensor proteins in neurons. SNAP25 is known as a protein associated with membrane fusion between a synaptic vesicle and a cell membrane. GRIA1, GRIA2, SYT1 and UCHL1 are known as membrane proteins expressed in neurons. Aβ is a polypeptide produced by cleavage of an amyloid-beta precursor protein (APP), and is known as a substance that accumulates in the brain of a patient with Alzheimer's dementia. Phosphorylated tau is a hyperphosphorylated tau protein, and is known as a causative substance of neurofibrillary tangle in the brain of a patient with Alzheimer's dementia. In the present specification, the term “Aβ” includes Aβ40 peptide, Aβ42 peptide, and Aβ oligomer. The Aβ oligomer refers to a multimer formed by physically or chemically polymerizing or aggregating multiple monomeric Aβ peptides. Since EVs generally contain proteins from cells from which they are derived, it is considered that the above proteins are also present in NDEVs. As shown in Examples described later, VILIP1, SYT1, UCHL1, GRIA1, SNAP25, GRIA2, SYT1, and UCHL1 were detected in EVs captured by the capture body containing TTC. This suggests that the EVs captured by the capture body containing TTC are NDEVs. In addition, it is known that Aβ and phosphorylated tau are mainly present in neurons. In the present embodiment, VILIP1, SYT1, UCHL1, GRIA1, SNAP25, GRIA2, SYT1, UCHL1, Aβ and phosphorylated tau can be used as neuron-specific markers.

The detector may be a substance capable of specifically binding to the target molecule of NDEV. Such a substance can be determined depending on the type of the target molecule, and may be, for example, an antibody, an aptamer, an oligonucleotide, a ligand receptor, a lipid receptor, a lectin, or the like. When the target molecule is a protein, the detector is preferably an antibody that specifically binds to the target molecule.

In the present specification, the term “antibody” also includes an antibody fragment. Examples of the antibody fragment include Fab, Fab′, F(ab′)2, and the like. The antibody may be either a monoclonal antibody or a polyclonal antibody. The origin of the antibody is not particularly limited, and may be an antibody derived from any mammal such as a mouse, a rat, a hamster, a rabbit, a goat, a horse, or a camel. The isotype of antibody may be any of IgG, IgM, IgE, IgA and the like and is preferably IgG.

The labeling substance is not particularly limited. Examples thereof include substances which themselves generate a signal (hereinafter also referred to as “signal generating substance”), substances which catalyze the reaction of other substances to generate a signal, and the like. Examples of the signal generating substance include fluorescent substances, radioactive isotopes, and the like. Examples of the substance which catalyzes a reaction of other substances to generate a detectable signal include enzymes. Examples of the enzymes include peroxidase, alkaline phosphatase, β-galactosidase, luciferase, and the like. Examples of the fluorescent substances include fluorescent dyes such as fluorescein isothiocyanate (FITC), rhodamine and Alexa Fluor (registered trademark), fluorescent proteins such as GFP, and the like. Examples of the radioactive isotopes include ¹²⁵I, ¹⁴C, ³²P, and the like. As the labeling substance, an enzyme is preferable, and peroxidase and alkaline phosphatase are particularly preferable.

As described above, the labeling substance is already contained in the detector or specifically bound to the detector. Thereby, in the complex, NDEV is labeled with the labeling substance via the detector. Examples of the case where the labeling substance is contained in the detector include using a detector containing the labeling substance. Such a detector is, for example, a detector to which the labeling substance is directly or indirectly bound (also referred to as a labeled detector). Examples of the detector to which the labeling substance is directly bound include a fusion protein of the above enzyme and antibody. Examples of the detector to which the labeling substance is indirectly bound include a detector to which the labeling substance is covalently bound via a linker. The labeling substance may be bound to the detector using a commercially available labeling kit.

Examples of the case where the labeling substance is specifically bound to the detector include using a substance that is labeled with the labeling substance and specifically binds to the detector. Such a substance is, for example, a labeled antibody that specifically binds to the detector. When the detector is an antibody, the labeled antibody that specifically binds to the detector is also called a labeled secondary antibody.

The solid phase may be any insoluble carrier capable of immobilizing the capture body. For example, the capture body can be immobilized on the solid phase by directly or indirectly binding the solid phase and the capture body. Examples of the direct binding between the solid phase and the capture body include physical adsorption or covalent bond to the surface of the solid phase. Examples of the indirect binding between the solid phase and the capture body include a covalent bond by a crosslinking agent. When the capture body is TTC to which the affinity tag is added, the solid phase and the capture body can be indirectly bound by using a solid phase on which a substance that binds to the affinity tag is immobilized.

The combination of an affinity tag and a substance that binds thereto is known per se. Examples of the combination of substances include combinations of any of biotin and its analogs and any of biotin-binding sites, a hapten and an anti-hapten antibody, a peptide tag and a substance that specifically binds to the tag, and the like. The biotin-binding sites include avidin and avidin analogs such as streptavidin and tamavidin (registered trademark). When the hapten is a DNP group, examples of the anti-hapten antibody include an anti-DNP antibody. Examples of the substance that specifically binds to the peptide tag include an antibody and an aptamer. When the peptide tag is a histidine tag, examples of a substance that specifically binds to the tag include Ni-NTA (nitrilotriacetic acid that forms a chelate with a nickel ion).

The material of the solid phase is not particularly limited. For example, the material can be selected from organic polymer compounds, inorganic compounds, biopolymers, and the like. Examples of the organic polymer compound include latex, polystyrene, polypropylene, and the like. Examples of the inorganic compound include magnetic bodies (iron oxide, chromium oxide, ferrite, and the like), silica, alumina, glass, and the like. Examples of the biopolymer include insoluble agarose, insoluble dextran, gelatin, cellulose, and the like. Two or more of these may be used in combination. The shape of the solid phase is not particularly limited, and examples thereof include a particle, a membrane, a microplate, a microtube, a test tube, and the like. Among them, a particle and a microplate are preferable. The particle is particularly preferably a magnetic particle.

Formation of the complex on the solid phase can be performed by mixing the capture body containing TTC, the biological sample, and the detector. The order of mixing is not particularly limited, and these may be mixed at a time or sequentially mixed. In a preferred embodiment, first, the capture body containing TTC and the biological sample are mixed (hereinafter, also referred to as “first mixing”). Next, the mixture of the capture body and the biological sample is mixed with the detector (hereinafter, also referred to as “second mixing”). The conditions of temperature and time in each mixing are not particularly limited. For example, the mixture is incubated at 4° C. to 40° C., and preferably at room temperature (about 20° C.) to 37° C., for 10 minutes to 24 hours, and preferably 20 minutes to 4 hours. During the incubation, the mixture may be allowed to stand, or may be stirred or shaken.

In the first mixing, NDEV in the biological sample and the capture body come into contact with each other, whereby the capture body and NDEV are bound to each other. In the first mixing, the capture body and the solid phase may come into contact with each other using the solid phase. For example, when the solid phase is a particle, the capture body containing TTC, the biological sample, and the solid phase are mixed. When the solid phase is a container such as a microplate, the capture body containing TTC and the biological sample are mixed in the container. Alternatively, the capture body containing TTC may be previously immobilized on the solid phase. As a result, NDEV is fixed onto the solid phase via the capture body.

After mixing the capture body and the biological sample, B/F (Bound/Free) separation for removing unreacted free components may be performed before further mixing the detector. The unreacted free components in the mixture of the capture body and the biological sample refers to, for example, components that are not bound to the capture body. Such components are, among components contained in the biological sample, cells that do not bind to TTC, EVs that do not bind to TTC, substances that do not bind to TTC (proteins, nucleic acids, lipids, sugar chains, and the like), and the like. By B/F separation, the capture body bound to NDEV can be selectively collected. The method of B/F separation is not particularly limited, but when the capture body is TTC immobilized on a particle, B/F separation can be performed by precipitating the particle by centrifugation and removing a supernatant containing an unreacted free component. When the particle is a magnetic particle, B/F separation can be performed, for example, by removing a liquid containing an unreacted free component while magnetically constraining the magnetic particle with a magnet, which is preferable from the viewpoint of automation. When the capture body is TTC immobilized in a container such as a microplate, B/F separation can be performed by removing a liquid containing an unreacted free component from the container. After B/F separation, the capture body bound to NDEV may be washed with a suitable aqueous medium such as PBS.

In the second mixing, the NDEV to which the capture body is bound comes into contact with the detector, whereby the target molecule of NDEV and the detector are bound. When the detector contains the labeling substance, NDEV is labeled with the labeling substance via binding between the target molecule and the detector. When the labeling substance is, for example, a labeled antibody, the mixture of the capture body and the biological sample is mixed with the detector, and then the labeled antibody is further mixed with the mixture. As a result, the labeled antibody specifically binds to the detector, and NDEV is indirectly labeled with the labeling substance.

After the complex is formed, B/F separation may be performed before a signal described later is detected. By B/F separation, unreacted free components that do not form a complex can be removed. Such a component is, for example, a detector that has not bound to the target molecule, a labeled antibody that has not bound to the detector, or the like. After B/F separation, the complex formed on the solid phase may be washed with a suitable aqueous medium such as PBS.

Through the first and second mixing, a complex containing the capture body, NDEV, the detector, and the labeling substance is formed on the solid phase. The structure of the complex will be described. In this complex, NDEV binds to the capture body via binding between ganglioside of the NDEV and TTC. Since the capture body is immobilized on the solid phase, NDEV is fixed onto the solid phase via the capture body. In addition, the captured NDEV is bound to the detector via the target molecule of the NDEV. Since the detector contains a labeling substance or is bound to the labeling substance, the captured NDEV is labeled with the labeling substance via the detector.

Next, a signal generated by the labeling substance contained in the complex is detected. The phrase “detecting a signal” herein includes qualitatively detecting the presence or absence of a signal, quantifying a signal intensity, and semi-quantitatively detecting the signal intensity. Semi-quantitative detection means to detect the signal intensity in a plurality of stages such as “no signal generated”, “weak”, “strong”, and the like. In a preferred embodiment, the signal intensity generated by the labeling substance contained in the complex is quantified.

In the present embodiment, EVs having the target molecule are measured based on the detected signal. The signal generated by the labeling substance contained in the complex indicates the presence of EV and/or the target molecule in the complex. For example, when the target molecule to which the detector is bound is an EV marker such as CD9, CD63, or CD81, the signal intensity reflects the amount of captured EV. Here, in view of the fact that TTC selectively binds to gangliosides GD1b and GT1b and that a neuron specific marker was detected in EVs captured by TTC, the signal intensity derived from the EV marker reflects the amount of NDEV. Therefore, when the target molecule is an EV marker, the measured value of the signal intensity can be used as the measured value of NDEV. In addition, when the target molecule to which the detector is bound is a neuron-specific marker such as VILIP1, SYT1, UCHL1, SNAP25, GRIA1, GRIA2, Aβ, or phosphorylated tau, the measured value of the signal intensity reflects the amount of the target molecule of the captured EVs. Therefore, when the target molecule is a neuron-specific marker, the measured value of the signal intensity can be used as the measured value of the target molecule, that is, the measured value of the neuron-specific marker.

A method for detecting a signal is known per se. In the present embodiment, a measurement method according to the type of signal derived from the labeling substance is appropriately selected. For example, when the labeling substance is an enzyme, signals such as light and color generated by reacting a substrate for the enzyme can be measured by using a known apparatus such as a spectrophotometer.

The substrate of the enzyme can be appropriately selected from known substrates according to the type of the enzyme. For example, when peroxidase is used as the enzyme, examples of the substrate include chemiluminescent substrates such as luminol and derivatives thereof, and chromogenic substrates such as 2,2′-azinobis(3-ethylbenzothiazoline-6-ammonium sulfonate) (ABTS), 1,2-phenylenediamine (OPD) and 3,3′,5,5′-tetramethylbenzidine (TMB). When alkaline phosphatase is used as the enzyme, examples of the substrate include chemiluminescent substrates such as CDP-Star (registered trademark) (disodium 4-chloro-3-(methoxyspiro[1,2-dioxetane-3,2′-(5′-chloro)tricyclo[3.3.1.13,7]decan]-4-yl)phenyl phosphate) and CSPD (registered trademark) (disodium 3-(4-methoxyspiro[1,2-dioxetane-3,2-(5′-chloro)tricyclo[3.3.1.13,7]decan]-4-yl)phenyl phosphate), and chromogenic substrates such as 5-bromo-4-chloro-3-indolyl phosphate (BCIP), disodium 5-bromo-6-chloro-indolyl phosphate and p-nitrophenyl phosphate. A commercially available substrate may be used.

When the labeling substance is a radioactive isotope, radiation as a signal can be measured using a known apparatus such as a scintillation counter. Also, when the labeling substance is a fluorescent substance, fluorescence as a signal can be measured using a known apparatus such as a fluorescence microplate reader. The excitation wavelength and the fluorescence wavelength can be appropriately determined according to the type of fluorescent substance used.

If necessary, a value obtained by subtracting the background value from the measured value of the signal intensity and the like can be used. Examples of the background value include measured values of signal intensity obtained by measurement without using any one of the biological sample, the capture body containing TTC, and the detector.

Another embodiment is a method for acquiring information on neurodegeneration of a subject. Hereinafter, this method is also referred to as “the method for acquiring information of the present embodiment”. Here, the neurodegeneration means degeneration that causes an abnormality in the structure and/or function of neurons, and causes a neurodegenerative disease. Examples of the neurodegenerative disease include Alzheimer's dementia (also referred to as Alzheimer's disease), Parkinson's disease, Huntington's disease, amyotrophic lateral sclerosis, multiple sclerosis, and the like. The measured value of the neuron specific marker can be used as an indicator of neurodegeneration of the subject. For example, the measured value of the neuron specific marker can be used as an indicator for determining the presence or absence of a neurodegenerative disease, the risk of contracting a neurodegenerative disease, the presence or absence of symptoms caused by a neurodegenerative disease, the risk of decline in cognitive function, and/or the state of cognitive function in the subject.

In the method for acquiring information of the present embodiment, the complex containing the capture body containing TTC, NDEV, the detector that specifically binds to the target molecule of NDEV, and the labeling substance is formed on the solid phase in vitro. Then, a signal generated by the labeling substance contained in the complex is detected in vitro. Details of the formation of the complex and the detection of the signal are the same as those described for the method for measuring EVs of the present embodiment. In the present embodiment, the target molecule is a neuron-specific marker, and examples thereof include VILIP1, SYT1, UCHL1, SNAP25, GRIA1, GRIA2, Aβ, phosphorylated tau, and the like. The number of target molecules may be one or two or more.

In the method for acquiring information of the present embodiment, signal detection is performed by quantifying signal intensity and acquiring a measured value. The acquired measured value of the signal can be an indicator of neurodegeneration of the subject. For example, the acquired measured value of the signal may indicate, as an indicator of neurodegeneration of the subject, the presence or absence of a neurodegenerative disease, the risk of contracting a neurodegenerative disease, the presence or absence of symptoms caused by a neurodegenerative disease, the risk of decline in cognitive function, and/or the state of cognitive function in the subject. In a preferred embodiment, the acquired measured value of the signal is compared with a predetermined threshold, and information on neurodegeneration of the subject is acquired based on the comparison result. Examples of such information include information indicating that the subject has or does not have a neurodegenerative disease, information indicating that the subject has a high or low risk of contracting a neurodegenerative disease, information indicating that the subject has or does not have a symptom caused by a neurodegenerative disease, information indicating that the subject has a high or low risk of declining cognitive function, and information indicating that the cognitive function of the subject has declined or has not declined. In a case where the subject is a patient with a neurodegenerative disease who has received a predetermined treatment for the neurodegenerative disease (hereinafter, it is also referred to as a “patient who has received a predetermined treatment”), information indicating that the neurodegenerative disease of the subject has been improved or not by the predetermined treatment, information indicating that the progress of the neurodegenerative disease of the subject is suppressed or not suppressed, and the like may be acquired as the information regarding the neurodegeneration of the subject. The predetermined threshold may be a threshold corresponding to each target molecule.

When the measured value of the signal for one target molecule is acquired, the signal measured value is compared with the threshold corresponding to the target molecule to acquire information on neurodegeneration of the subject. As an example, a case where one target molecule is VILIP1 will be described. When the signal measured value of VILIP1 is lower than the threshold corresponding to VILIP1, at least one of information indicating that the subject has a neurodegenerative disease, information indicating that the subject has a high risk of contracting a neurodegenerative disease, information indicating that the subject has a symptom caused by a neurodegenerative disease, information indicating that the subject has a high risk of declining cognitive function, and information indicating that the cognitive function of the subject has declined is acquired. When the signal measured value of VILIP1 is equal to or greater than the threshold corresponding to VILIP1, at least one of information indicating that the subject does not have a neurodegenerative disease, information indicating that the subject has a low risk of contracting a neurodegenerative disease, information indicating that the subject does not have a symptom caused by a neurodegenerative disease, information indicating that the subject has a low risk of declining cognitive function, and information indicating that the cognitive function of the subject has not declined is acquired. When the subject is a patient who has received a predetermined treatment and the signal measured value of VILIP1 is lower than the threshold corresponding to VILIP1, at least one of information indicating that the neurodegenerative disease of the subject is not improved by the predetermined treatment and information indicating that the progress of neurodegeneration of the subject is not suppressed by the predetermined treatment is acquired. When the subject is a patient who has received a predetermined treatment and the signal measured value of VILIP1 is equal to or greater than the threshold corresponding to VILIP1, at least one of information indicating that the neurodegenerative disease of the subject is improved by the predetermined treatment and information indicating that the progress of neurodegeneration of the subject is suppressed by the predetermined treatment is acquired. Here, an example in which one target molecule is VILIP1 has been described, but the present invention is not limited to this example. Instead of the signal measured value of VILIP1, the signal measured value for SYT1, UCHL1, SNAP25, GRIA1, GRIA2, Aβ or phosphorylated tau may be used. In this case, a threshold corresponding to the selected target molecule is used instead of the threshold corresponding to VILIP1.

In one embodiment, the target molecule includes at least two selected from a group consisting of VILIP1, SYT1, UCHL1, SNAP25, GRIA1, GRIA2, Aβ, and phosphorylated tau, and information on neurodegeneration of the subject may be acquired based on the signal measured values thereof. For example, when at least one of the signal measured values of the target molecules selected from the above group is lower than the threshold corresponding to the target molecule, at least one of information indicating that the subject has a neurodegenerative disease, information indicating that the subject has a high risk of contracting a neurodegenerative disease, information indicating that the subject has a symptom caused by a neurodegenerative disease, information indicating that the subject has a high risk of declining cognitive function, and information indicating that the cognitive function of the subject has declined is acquired. For example, when all of the signal measured values of the target molecules selected from the above group are equal to or more than the threshold corresponding to each target molecule, at least one of information indicating that the subject does not have a neurodegenerative disease, information indicating that the subject has a low risk of contracting a neurodegenerative disease, information indicating that the subject does not have a symptom caused by a neurodegenerative disease, information indicating that the subject has a low risk of declining cognitive function, and information indicating that the cognitive function of the subject does not decline is acquired. For example, when the subject is a patient who has received a predetermined treatment and at least one of the signal measured values of the target molecules selected from the above group is lower than the threshold corresponding to the target molecule, at least one of information indicating that the neurodegenerative disease of the subject is not improved by the predetermined treatment and information indicating that the progress of neurodegeneration of the subject is not suppressed by the predetermined treatment is acquired. For example, when the subject is a patient who has received a predetermined treatment and all of the signal measured values of the target molecules selected from the above group are equal to or more than the threshold corresponding to the target molecule, at least one of information indicating that the neurodegenerative disease of the subject is improved by the predetermined treatment and information indicating that the progress of neurodegeneration of the subject is suppressed by the predetermined treatment is acquired.

As an example, when the target molecules are two selected from the group consisting of VILIP1, SYT1, UCHL1, SNAP25, GRIA1, GRIA2, Aβ, and phosphorylated tau, and at least one of the signal measured values thereof is lower than the threshold corresponding to the target molecule, at least one of information indicating that the subject has a neurodegenerative disease, information indicating that the subject has a high risk of contracting a neurodegenerative disease, information indicating that the subject has a symptom caused by a neurodegenerative disease, information indicating that the subject has a high risk of declining cognitive function, and information indicating that the cognitive function of the subject has declined (hereinafter, these information are also referred to as “information disadvantageous to the subject with respect to neurodegeneration”) is acquired. In another example, when the target molecules are three selected from the group consisting of VILIP1, SYT1, UCHL1, SNAP25, GRIA1, GRIA2, Aβ, and phosphorylated tau, and at least one of the signal measured values thereof is lower than the threshold corresponding to the target molecule, at least one of information disadvantageous to the subject with respect to neurodegeneration is acquired. In another example, when the target molecules are four selected from the group consisting of VILIP1, SYT1, UCHL1, SNAP25, GRIA1, GRIA2, Aβ, and phosphorylated tau, and at least one of the signal measured values thereof is lower than the threshold corresponding to the target molecule, at least one of information disadvantageous to the subject with respect to neurodegeneration is acquired. In another example, when the target molecules are five selected from the group consisting of VILIP1, SYT1, UCHL1, SNAP25, GRIA1, GRIA2, Aβ, and phosphorylated tau, and at least one signal measured value of the signal measured values thereof is lower than the threshold corresponding to the target molecule, at least one of information disadvantageous to the subject with respect to neurodegeneration is acquired. In another example, when the target molecules are six selected from the group consisting of VILIP1, SYT1, UCHL1, SNAP25, GRIA1, GRIA2, Aβ, and phosphorylated tau, and at least one signal measured value of the signal measured values thereof is lower than the threshold corresponding to the target molecule, at least one of information disadvantageous to the subject with respect to neurodegeneration is acquired. In another example, when the target molecules are seven selected from the group consisting of VILIP1, SYT1, UCHL1, SNAP25, GRIA1, GRIA2, Aβ, and phosphorylated tau, and at least one signal measured value of the signal measured values thereof is lower than the threshold corresponding to the target molecule, at least one of information disadvantageous to the subject with respect to neurodegeneration is acquired. In another example, when the target molecules are VILIP1, SYT1, UCHL1, SNAP25, GRIA1, GRIA2, Aβ, and phosphorylated tau, and at least one of the signal measured values thereof is lower than the threshold corresponding to the target molecule, at least one of information disadvantageous to the subject with respect to neurodegeneration is acquired.

A case where the subject is a patient who has received a predetermined treatment will be exemplified. For example, when the target molecules are two selected from the group consisting of VILIP1, SYT1, UCHL1, SNAP25, GRIA1, GRIA2, Aβ, and phosphorylated tau, and at least one of the signal measured values thereof is lower than the threshold corresponding to the target molecule, at least one of information indicating that the neurodegenerative disease of the subject is not improved by the predetermined treatment and information indicating that the progress of neurodegeneration of the subject is not suppressed by the predetermined treatment (hereinafter, these information are also referred to as “information disadvantageous to the subject with respect to therapeutic effect”) is acquired. In another example, when the target molecules are three selected from the group consisting of VILIP1, SYT1, UCHL1, SNAP25, GRIA1, GRIA2, Aβ, and phosphorylated tau, and at least one of the signal measured values thereof is lower than the threshold corresponding to the target molecule, at least one of information disadvantageous to the subject with respect to therapeutic effect is acquired. In another example, when the target molecules are four selected from the group consisting of VILIP1, SYT1, UCHL1, SNAP25, GRIA1, GRIA2, Aβ, and phosphorylated tau, and at least one of the signal measured values thereof is lower than the threshold corresponding to the target molecule, at least one of information disadvantageous to the subject with respect to therapeutic effect is acquired. In another example, when the target molecules are five selected from the group consisting of VILIP1, SYT1, UCHL1, SNAP25, GRIA1, GRIA2, Aβ, and phosphorylated tau, and at least one signal measured value of the signal measured values thereof is lower than the threshold corresponding to the target molecule, at least one of information disadvantageous to the subject with respect to therapeutic effect is acquired. In another example, when the target molecules are six selected from the group consisting of VILIP1, SYT1, UCHL1, SNAP25, GRIA1, GRIA2, Aβ, and phosphorylated tau, and at least one signal measured value of the signal measured values thereof is lower than the threshold corresponding to the target molecule, at least one of information disadvantageous to the subject with respect to therapeutic effect is acquired. In another example, when the target molecules are seven selected from the group consisting of VILIP1, SYT1, UCHL1, SNAP25, GRIA1, GRIA2, Aβ, and phosphorylated tau, and at least one signal measured value of the signal measured values thereof is lower than the threshold corresponding to the target molecule, at least one of information disadvantageous to the subject with respect to therapeutic effect is acquired. In another example, when the target molecules are VILIP1, SYT1, UCHL1, SNAP25, GRIA1, GRIA2, Aβ, and phosphorylated tau, and at least one of the signal measured values thereof is lower than the threshold corresponding to the target molecule, at least one of information disadvantageous to the subject with respect to therapeutic effect is acquired.

When the target molecules are at least two selected from the group consisting of VILIP1, SYT1, UCHL1, SNAP25, GRIA1, GRIA2, Aβ, and phosphorylated tau, information in which the risk related to neurodegeneration of the subject and the like are classified into three stages may be acquired from the signal measured values thereof. For example, when all of the signal measured values of the target molecules selected from the above group are lower than the threshold corresponding to each target molecule, at least one of information indicating that the subject has a neurodegenerative disease, information indicating that the subject has a high risk of contracting a neurodegenerative disease, information indicating that the subject has a symptom caused by a neurodegenerative disease, information indicating that the subject has a high risk of declining cognitive function, and information indicating that the cognitive function of the subject has declined is acquired. When at least one of the signal measured values of the target molecules selected from the above group is lower than the threshold corresponding to the target molecule, at least one of information indicating that the possibility that the subject has a neurodegenerative disease is moderate, information indicating that the subject has a moderate risk of contracting a neurodegenerative disease, information indicating that the possibility that the subject has a symptom caused by a neurodegenerative disease is moderate, information indicating that the subject has a moderate risk of declining cognitive function, and information indicating that the possibility that the cognitive function of the subject has declined is moderate is acquired. When all of the signal measured values of the target molecules selected from the above group are equal to or more than the threshold corresponding to each target molecule, at least one of information indicating that the subject does not have a neurodegenerative disease, information indicating that the subject has a low risk of contracting a neurodegenerative disease, information indicating that the subject does not have a symptom caused by a neurodegenerative disease, information indicating that the subject has a low risk of declining cognitive function, and information indicating that the cognitive function of the subject does not decline is acquired.

When the subject is a patient who has received a predetermined treatment and the target molecules are at least two selected from the group consisting of VILIP1, SYT1, UCHL1, SNAP25, GRIA1, GRIA2, Aβ, and phosphorylated tau, information in which the effect of the predetermined treatment for the neurodegenerative disease is classified into three stages may be acquired from the signal measured values thereof. For example, when all of the signal measured values of the target molecules selected from the above group are lower than the threshold corresponding to each target molecule, information indicating that improvement of neurodegenerative disease by the predetermined treatment and/or suppression of the progress of neurodegeneration by the predetermined treatment is “−” is acquired. When any one of the signal measured values of the target molecules selected from the above group is equal to or more than the threshold corresponding to the target molecule, information indicating that improvement of neurodegenerative disease by the predetermined treatment and/or suppression of the progress of neurodegeneration by the predetermined treatment is “+” is acquired. For example, when all of the signal measured values of the target molecules selected from the above group are equal to or more than the threshold corresponding to the target molecule, information indicating that improvement of neurodegenerative disease by the predetermined treatment and/or suppression of the progress of neurodegeneration by the predetermined treatment is “++” is acquired. Here, the above “−” indicates that improvement of neurodegenerative disease and/or suppression of the progress of neurodegeneration is not observed, and the above “+” and “++” indicate that improvement of neurodegenerative disease and/or suppression of the progress of neurodegeneration is observed. In addition, the above “++” indicates that the neurodegenerative disease is more improved and/or the progress of neurodegeneration is more suppressed than the “+”.

The predetermined threshold is not particularly limited. The predetermined threshold can be appropriately set. For example, NDEVs in biological samples obtained from each of a plurality of healthy subjects (normal group) and a plurality of subjects having a neurodegenerative disease or a symptom caused by the neurodegenerative disease (abnormal group) are measured to obtain data of signal measured values of target molecules. Then, a value capable of discriminating between the normal group and the abnormal group with the highest accuracy is obtained, and the value is set as a predetermined threshold. In setting the threshold, it is preferable to consider sensitivity, specificity, positive predictive value, negative predictive value, and the like.

A medical worker such as a doctor can use the acquired signal measured value as an indicator for determining the state of neurodegeneration and/or cognitive function of the subject. In addition, the signal measured value and other information may be combined to determine the state of neurodegeneration and/or cognitive function of the subject. The term “other information” herein includes, for example, information obtained by Mini-Mental State Examination (MMSE), measurement of Aβ40, Aβ42, tau protein (total tau, phosphorylated tau) and the like in CSF or the blood sample, diagnostic imaging of Aβ or tau protein by PET, and the like, and other medical findings.

When information disadvantageous to the subject with respect to neurodegeneration is acquired, treatment for neurodegeneration and/or cognitive decline of the subject may be performed. For example, a drug is administered to the subject. Examples of the drug include donepezil, galantamine, rivastigmine, memantine, aducanumab, and the like.

A further embodiment relates to a method for isolating NDEVs in a biological sample in vitro. Hereinafter, this method is also referred to as “the method for isolating EVs of the present embodiment”. In the method for isolating EVs of the present embodiment, first, a capture body containing TTC and NDEV are bound in vitro. This can be performed by mixing the capture body containing TTC and the biological sample. This mixing contacts NDEV in the biological sample with the capture body. At this time, it is considered that TTC in the capture body can bind to GD1b and/or GT1b on the surface of NDEVs to selectively capture NDEV in the biological sample. The conditions of temperature and time in mixing are as described above.

In the present embodiment, it is preferable to mix the solid phase on which the capture body is immobilized with the biological sample. By this mixing, the capture body and NDEV are bound on the solid phase. That is, NDEV is fixed onto the solid phase via the capture body. Accordingly, the captured NDEVs can be efficiently collected. Details of the solid phase are the same as those described for the method for measuring EVs of the present embodiment.

In the present embodiment, unreacted free components not bound to the capture body are removed. This can be performed by the B/F separation described above. The unreacted free components not bound to the capture body are, among components contained in the biological sample, for example, cells that do not bind to TTC, EVs that do not bind to TTC, substances that do not bind to TTC (proteins, nucleic acids, lipids, sugar chains, and the like), and the like. By removing the unreacted free components, the capture body bound to NDEV is selectively collected. That is, NDEVs can be isolated from the biological sample. Details of the B/F separation are the same as those described for the method for measuring EVs of the present embodiment. After B/F separation, the capture body bound to NDEV may be washed with a suitable aqueous medium such as PBS.

NDEVs isolated by the capture body can be utilized in various assays. For example, the isolated NDEVs may be dissolved with a suitable solubilizer to analyze the contents. Such solubilizers include buffers containing a surfactant capable of lysing cells. The method for analyzing the contents is not particularly limited, and can be arbitrarily selected from known protein analysis methods, nucleic acid analysis methods, and the like. The isolated NDEVs may also be analyzed without destruction. Such an analysis method is not particularly limited, and examples thereof include a solid-phase ligand binding assay, observation with an electron microscope, and the like.

A further embodiment relates to a reagent kit containing a capture body containing TTC (hereinafter, also referred to as “the reagent kit of the present embodiment”). The reagent kit of the present embodiment is used in the method for measuring EVs of the present embodiment, the method for acquiring information on cognitive function of the present embodiment, and the method for isolating extracellular vesicles of the present embodiment. Details of the capture body containing TTC are the same as those described for the method for measuring EVs of the present embodiment.

The reagent kit of the present embodiment may further contain a solid phase. Alternatively, the reagent kit of the present embodiment may contain TTC immobilized on a solid phase. Details of the solid phase are the same as those described for the method for measuring EVs of the present embodiment. For example, when the solid phase is a particle, the reagent kit of the present embodiment contains a TTC-immobilized particle as a capture body. When the solid phase is a microplate, the reagent kit of the present embodiment contains a TTC-immobilized plate as a capture body.

The reagent kit of the present embodiment may further contain a detector that specifically binds to the target molecule of NDEV. The detector may contain a labeling substance. Alternatively, the reagent kit of the present embodiment may further contain a detector that specifically binds to the target molecule of NDEV and a labeling substance. When the labeling substance is an enzyme, the reagent kit of the present embodiment may further contain a substrate for the enzyme. It is preferable that the capture body, the detector, the labeling substance and the substrate be stored in separate containers or individually packaged. Details of the detector, the labeling substance and the substrate are the same as those described for the method for measuring EVs of the present embodiment.

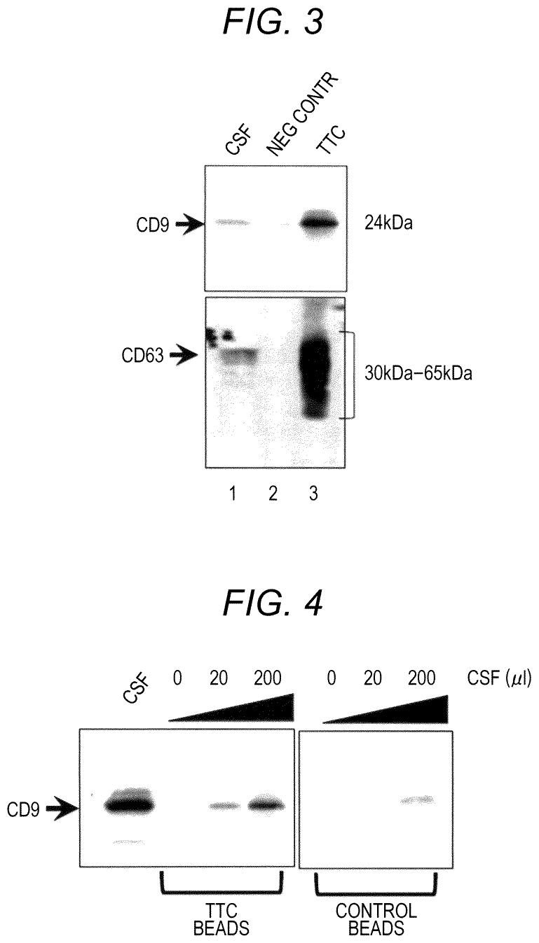

The reagent kit may be provided to a user by packing a container containing each reagent in a box. The box may contain an attached document. Configuration of the reagent kit, composition of each reagent, usage and the like may be described in the attached document. FIG. 2A shows an example of the reagent kit of the present embodiment. With reference to FIG. 2A, 11 denotes a reagent kit, 12 denotes a container containing a reagent containing a capture body, 13 denotes a packing box, and 14 denotes a package insert. The capture body in the reagent may be immobilized on a particle, preferably a magnetic particle.

FIG. 2B shows an example of a reagent kit of the present embodiment further containing a detector. Referring to FIG. 2B, 2I denotes a reagent kit, 22 denotes a first container containing a first reagent containing a capture body, 23 denotes a second container containing a second reagent containing a detector, 24 denotes a packing box, and 25 denotes an attached document. The detector in the reagent may be a labeled detector to which a labeling substance is bound.

It can also be said that the capture body containing TTC is used for the production of the reagent kit of the present embodiment. That is, another embodiment is a use of a capture body containing TTC for producing a reagent kit for use in a method for measuring extracellular vesicles. A further embodiment is a use of a capture body containing TTC for producing a reagent kit for use in a method for acquiring information on neurodegeneration. A further embodiment is a use of a capture body containing TTC for producing a reagent kit for use in a method for isolating extracellular vesicles. In the production of the reagent kit of the present embodiment, the above detector may be further used. A further embodiment is a use of a capture body containing TTC and a detector that specifically binds to a target molecule of extracellular vesicles for producing a reagent kit for use in a method for measuring extracellular vesicles. A further embodiment is a use of a capture body containing TTC and a detector that specifically binds to a target molecule of extracellular vesicles for producing of a reagent kit for use in a method for acquiring information on neurodegeneration. A further embodiment is a use of a capture body containing TTC and a detector that specifically binds to a target molecule of extracellular vesicles for producing a reagent kit for use in a method for isolating extracellular vesicles.

Hereinbelow, the present invention will be described in detail by examples, but the present invention is not limited to these examples.

### EXAMPLES

### Example 1: Capture of Extracellular Vesicles by Capture Body Containing Tetanus Toxin C-Terminal Fragment

Using TTC immobilized on a solid phase (magnetic particle) as a capture body containing TTC, whether or not extracellular vesicles in a biological sample can be captured was examined.

### (1) Materials

As biological samples, human donor-derived pooled CSF (MyBioSource), pooled human plasma (K2-EDTA added) (Innovative Research), and pooled human plasma (Na-heparin added) (Innovative Research) were used. As TTC, recombinant tetanus toxin heavy chain fragment C (Fina Biosolutions) was used. As a solid phase, Dynabeads (trademark) M-270 epoxy (2.8 μm in diameter, superparamagnetic) (Thermo Fisher Scientific) was used. As an elution reagent, 2× Laemmli sample buffer (125 mM Tris-HCL, pH 6.8, 20% (w/v) glycerol, 4% SDS, 0.01% bromophenol blue) was used. As a blocking solution, Blocking One (Nacalai Tesque, Inc.) was used. As primary antibodies, an anti-CD9 mouse monoclonal antibody (Cosmobio and Biolegend) and an anti-CD63 mouse monoclonal antibody (Thermo Fisher Scientific) were used. As a secondary antibody, an HRP-labeled anti-mouse IgG Fc antibody (Novus Biologicals) was used.

### (2) Preparation of Capture Body Containing TTC

According to a manual for the magnetic particle, 10 μg of TTC per 1 mg of magnetic particles was bound to the particle surface. Specifically, the magnetic particles, TTC, and a mixture of equal amounts of C1 buffer and C2 buffer were mixed and stirred at 37° C. overnight. The obtained TTC-immobilized particles were washed with PBS. Hereinafter, the TTC-immobilized particle is also referred to as a “capture body”.

### (3) Capture of EVs in Biological Samples

Predetermined amounts of CSF and plasma were centrifuged at 3000×g at 4° C. for 15 min and the respective supernatants were collected. Each supernatant was diluted with PBS containing complete (trademark) protease inhibitor cocktail (Roche) to 0.5 mL. The diluted supernatant (0.5 mL) and the capture body (1 mg) were mixed, and the mixture was stirred at room temperature for 3 hours. The capture body was separated with a magnetic stand and washed three times with PBS. After washing, the elution reagent (40 μL) was added to the capture body, and the mixture was incubated at 80° C. for 5 minutes to obtain an eluate. For comparison, an experiment was similarly performed using magnetic particles to which TTC is not bound to obtain an eluate. The eluate was filled in a Supercep (trademark) Ace 5 to 20% gel (FUJIFILM Wako Pure Chemical Corporation) and electrophoresed, and then the protein in the gel was transferred to a PVDF membrane. The membrane was blocked with a blocking solution at room temperature for 1 hour, and then incubated at 4° C. overnight in a solution of primary antibody diluted with 10% blocking solution/PBS. The membrane was washed three times with a HISCL (registered trademark) washing solution (Sysmex Corporation), and then incubated in a solution of secondary antibody diluted with 10% blocking solution/PBS at room temperature for 1 hour. The membrane was washed three times with a HISCL washing solution, and then a chemiluminescent signal was generated using an ECL (trademark) Prime Western Blotting System (GE Healthcare). The signal was detected with ImageQuant (trademark) LAS 4000 mini (cytiva).

### (4) Results

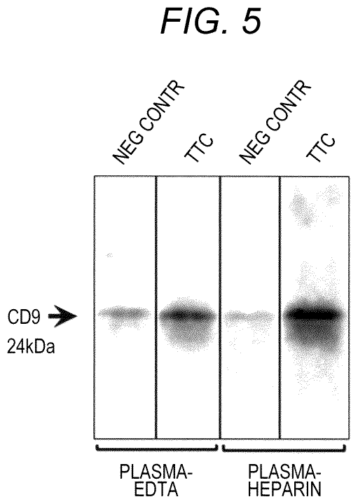

The results of an experiment using CSF as a biological sample are shown in FIG. 3. In FIG. 3, “CSF” (lane 1) indicates a lane in which CSF diluted with PBS was migrated, “Neg Contr” (lane 2) indicates a lane in which an eluate obtained using the magnetic particles to which TTC was not bound was migrated, and “TTC” (lane 3) indicates a lane in which an eluate obtained using a capture body (TTC) was migrated. In lane 3, bands of CD9 and CD63 known as exosome marker proteins appeared remarkably. On the other hand, in lane 2, bands of CD9 and CD63 were hardly observed. These results suggested that EVs in CSF could be captured by the TTC-immobilized particles.

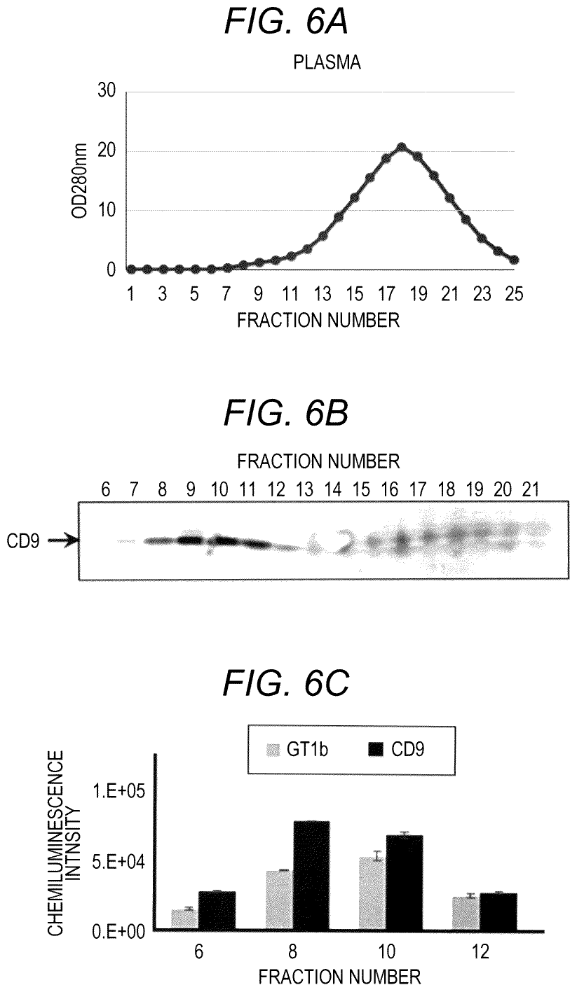

The results of an experiment using 0, 20, or 200 μL of CSF are shown in FIG. 4. In FIG. 4, CSF diluted with PBS was migrated in the lane of “CSF”. In the figure, “TTC-beads” in a left panel indicates a lane in which an eluate obtained using the capture body was migrated, and “Control beads” in a right panel indicates a lane in which an eluate obtained using magnetic particles to which TTC was not bound was migrated. As can be seen from the left panel of FIG. 4, the higher the amount of CSF used, the significantly darker the band of CD9. On the other hand, in the right panel of FIG. 4, the band of CD9 was hardly observed. From these results, it was suggested that the amount of EVs captured by the TTC-immobilized particles increases according to the amount of CSF.

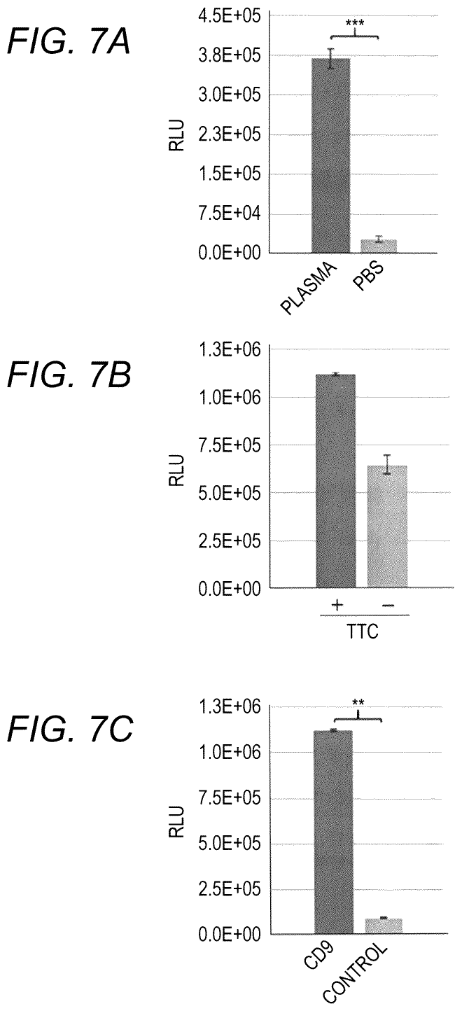

The results of an experiment using plasma as a biological sample are shown in FIG. 5. In the figure, “TTC” indicates a lane in which an eluate obtained using the capture body was migrated, and “Neg Contr” indicates a lane in which an eluate obtained using the magnetic particles to which TTC was not bound was migrated. “Plasma-EDTA” indicates that the biological sample was plasma to which EDTA was added, and “Plasma-Heparin” indicates that the biological sample was plasma to which heparin was added. Even when either plasma was used, the band of CD9 was remarkably observed in the lane of TTC as compared with the lane of Neg Contr. These results suggested that EVs in plasma could be captured by the TTC-immobilized particles.

### Example 2: Confirmation of GT1b Ganglioside in Extracellular Vesicle

TTC is known to selectively bind to GT1b ganglioside. Therefore, in order to examine whether the capture of EVs by TTC-immobilized particles depends on the binding between TTC and GT1b ganglioside, whether or not GT1b ganglioside was present in extracellular vesicles in a biological sample was confirmed.

### (1) Obtainment of Fraction Containing EV

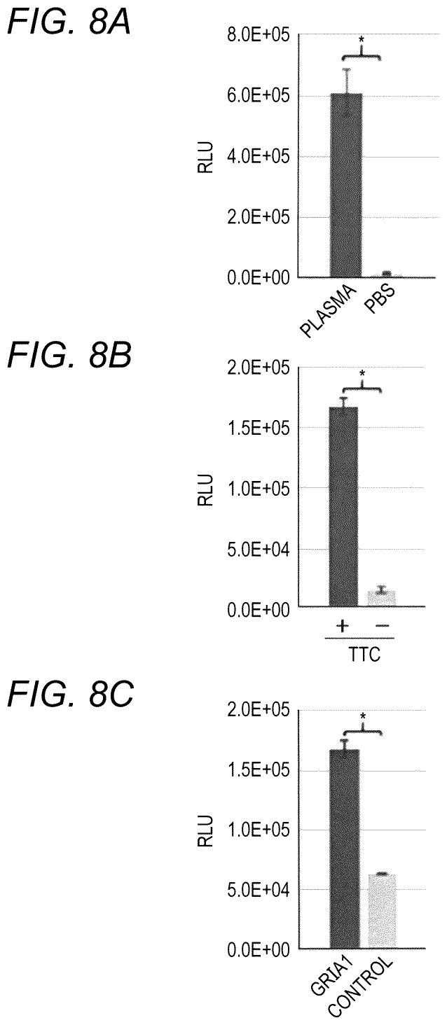

Pooled human plasma was separated by size exclusion chromatography to obtain a fraction containing EV. Specific operations are as follows. First, 10 mL of Sepharose (registered trademark) CL-4B resin (GE Healthcare) was packed in an Econo-Pac (registered trademark) chromatography column (Bio-Rad) and washed with PBS (50 mL). Pooled human plasma (1 mL) was loaded onto the column and then eluted with PBS. Fractions No. 1 to No. 25 were obtained by collecting 0.5 mL of an eluate. The protein content of each fraction was measured by absorbance at 280 nm using NanoDrop (trademark) 2000c (Thermo Fisher Scientific). The measurement results of the absorbance are shown in FIG. 6A. As shown in FIG. 6A, in the fractions No. 6 to No. 12, the absorbance at 280 nm slightly increased, and the absorbance at 280 nm rapidly increased from the fraction No. 13. This suggested that the fractions No. 6 to No. 12 contained EV and the fraction No. 13 and subsequent fractions contained a solubilized protein.

### (2) Confirmation of Presence of EV in Each Fraction

From the results of (1) above, whether the fractions No. 6 to No. 21 contained EVs was examined. Specifically, it was examined as follows. First, as samples, equal amounts of eluate were fractionated from each of the fractions No. 6 to No. 21. Proteins in each sample were separated by SDS-PAGE and subjected to Western blot analysis using an anti-CD9 antibody in the same manner as in Example 1. The results of Western blot analysis are shown in FIG. 6B. As shown in FIG. 6B, among the fractions No. 6 to No. 21, the band of CD9 could be observed in the fractions No. 7 to No. 12. This showed that the fractions of No. 7 to No. 12 contained EVs.

### (3) Confirmation of GT1b Ganglioside in EV

From the results of (2) above, the fractions of No. 6, No. 8, No. 10, and No. 12 were used as samples. Whether or not GT1b ganglioside was present in EVs in each fraction was examined by ELISA method. Specifically, it was examined as follows. A part of the eluate was collected from each fraction, added to each well of a Maxisorp (trademark) 96-well plate (Thermo Fisher Scientific), and the mixture was incubated at 4° C. overnight. The solution was removed from each well, and then the plate was washed three times with a HISCL washing solution. Blocking One (Nacalai Tesque, Inc.) was added to each well, and the plate was incubated at room temperature for 1 hour. After removing the solution from each well, a solution of an anti-CD9 mouse monoclonal antibody or an anti-GT1b mouse monoclonal antibody (Developmental Studies Hybridoma Bank) was added to each well, and the mixture was incubated at 37° C. for 1 hour. The solution was removed from each well, and then the plate was washed three times with a HISCL washing solution. A solution of HRP-labeled anti-mouse IgG Fc antibody (Novus Biologicals) was added to each well, and the mixture was incubated at 37° C. for 1 hour. The solution was removed from each well, and then the plate was washed three times with a HISCL washing solution. A BM chemiluminescent ELISA substrate (POD) (Roche) was added to each well to generate a chemiluminescent signal. The signal was detected with Infinite (registered trademark) F200 PRO (TECAN). The measurement results are shown in FIG. 6C.

As shown in FIG. 6C, CD9 was detected in all fractions. In particular, it was found that CD9 was contained in a large amount in the fractions No. 8 and No. 10. This result was similar to the Western blot analysis of FIG. 6B. Therefore, the ELISA method also showed that the fraction contained EVs. Similarly to CD9, GT1b was also detected in all fractions, and was contained in a large amount in the fractions No. 8 and No. 10. These showed that GT1b ganglioside was present on the surface of EVs in the fraction. Therefore, in Example 1, it was suggested that by mixing the biological sample and the TTC-immobilized particles, TTC and GT1b ganglioside on the EV surface bound to each other, and EVs were captured by the TTC-immobilized particles.

### Example 3: Detection of Extracellular Vesicles by Solid-Phase Ligand Binding Assay

It was examined whether or not extracellular vesicles in a biological sample can be detected by a solid-phase ligand binding assay using TTC immobilized on a solid phase (microplate) and an antibody against CD9 which is an exosome marker protein.

### (1) Solid-Phase Ligand Binding Assay

A solution of TTC solubilized in PBS (2 μg/mL) was added to each well of a Maxisorp (trademark) 96-well plate (Thermo Fisher Scientific), and the mixture was incubated at 4° C. overnight. The solution was removed from each well, and then the plate was washed three times with a HISCL washing solution. Blocking One (Nacalai Tesque, Inc.) was added to each well, and the plate was incubated at room temperature for 1 hour. After removing the solution from each well, pooled human plasma diluted 5-fold with a blocking solution was added to each well, and the mixture was incubated at room temperature for 2 hours. The solution was removed from each well, and then the plate was washed three times with a HISCL washing solution. A solution of anti-CD9 mouse monoclonal antibody was added to each well, and the plate was incubated at 37° C. for 1 hour. The solution was removed from each well, and then the plate was washed three times with a HISCL washing solution. A solution of HRP-labeled rabbit anti-mouse IgG Fc antibody (Novus Biologicals) was added to each well, and the plate was incubated at 37° C. for 1 hour. The solution was removed from each well, and then the plate was washed three times with a HISCL washing solution. A BM chemiluminescent ELISA substrate (POD) (Roche) was added to each well to generate a chemiluminescent signal. The signal was detected with Infinite (registered trademark) F200 PRO (TECAN). For comparison, an experiment in which PBS was added instead of plasma, an experiment using a plate on which TTC was not immobilized, and an experiment using mouse IgG2a (BioLegend) and mouse IgG2b (BD Bioscience) as isotype controls instead of the anti-CD9 antibody were also performed.

### (2) Results

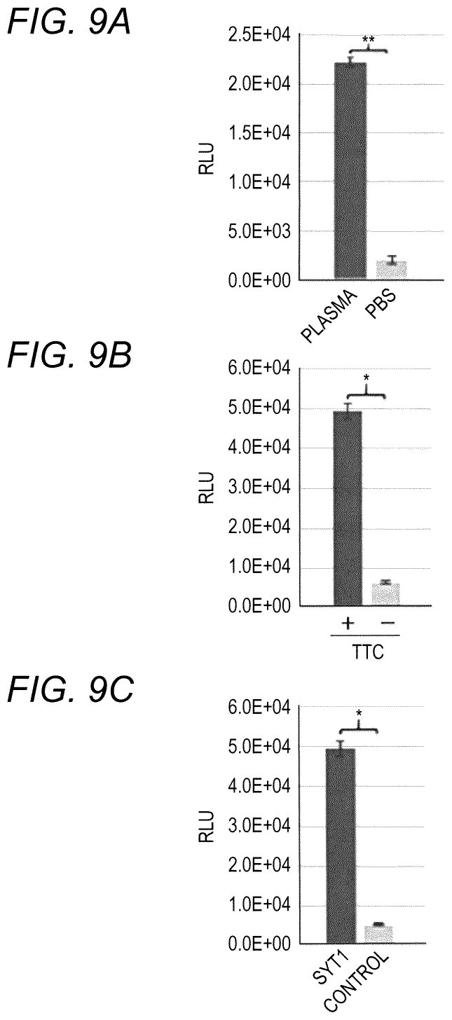

The measurement results are shown in FIGS. 7A to 7C. In the figure, “**” represents p≤0.01, “***” represents p≤0.001, “CD9” represents an anti-CD9 antibody, and “control” represents an isotype control. As shown in FIG. 7A, the signal of CD9 significantly increased in the case where plasma was added to the microplate as compared with the case where plasma was not added. As shown in FIG. 7B, even when the plate on which TTC was not immobilized was used, the signal of CD9 was detected. As shown in FIG. 7C, the signal significantly increased in the case of using the anti-CD9 antibody as compared with the case of using the isotype control. From these measurement results, it was shown that EVs in plasma can be captured by TTC, and the captured EVs can be detected with the anti-CD9 antibody. The result of FIG. 7B was considered to be caused by non-specific binding of EVs to the plate even when blocking occurred. However, it was shown that the signal of CD9 was sufficiently higher when the plate on which TTC was immobilized was used. This example showed that EVs in a biological sample can be highly specifically detected by a solid-phase ligand binding assay using TTC immobilized on a solid phase and an antibody against a surface antigen of EVs.

### Example 4: Detection of Neuron-Derived Extracellular Vesicles by Solid-Phase Ligand Binding Assay

It was examined whether or not neuron-derived extracellular vesicles in a biological sample can be detected by a solid-phase ligand binding assay using TTC immobilized on a solid phase (microplate) and an antibody against a neuron-specific marker.

### (1) Solid-Phase Ligand Binding Assay

A solid-phase ligand binding assay was performed in the same manner as in Example 3 except that a primary antibody and a secondary antibody described later were used. As primary antibodies, an anti-GRIA1 mouse monoclonal antibody (Novus Biologicals), and an anti-SYT1 rabbit polyclonal antibody (Proteintech) and an anti-UCHL1 rabbit polyclonal antibody (Proteintech) were used. As a secondary antibody against a mouse monoclonal antibody, the same HRP-labeled anti-mouse IgG Fc antibody as in Example 3 was used. As a secondary antibody against rabbit polyclonal antibodies, an HRP-labeled goat anti-rabbit IgG antibody (Cell Signaling Technology) was used. For comparison, an experiment in which PBS was added instead of plasma, an experiment using a plate on which TTC was not immobilized, and an experiment using mouse IgG2a (BioLegend) and rabbit IgG (BD Bioscience) as isotype controls instead of various primary antibodies were also performed.

### (2) Results

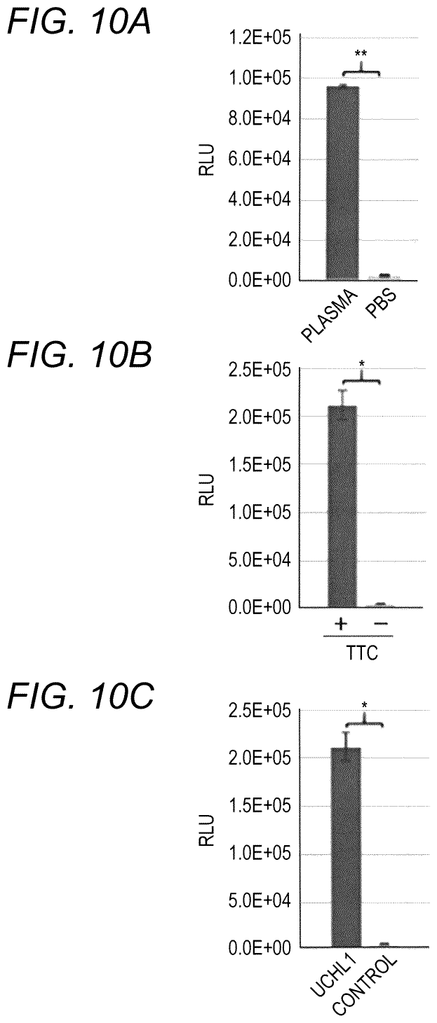

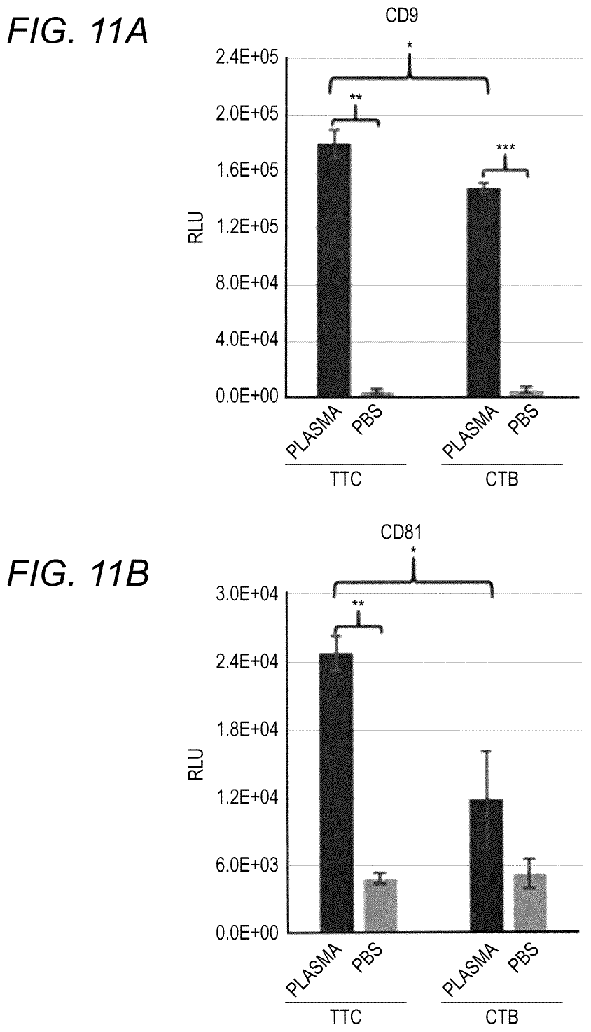

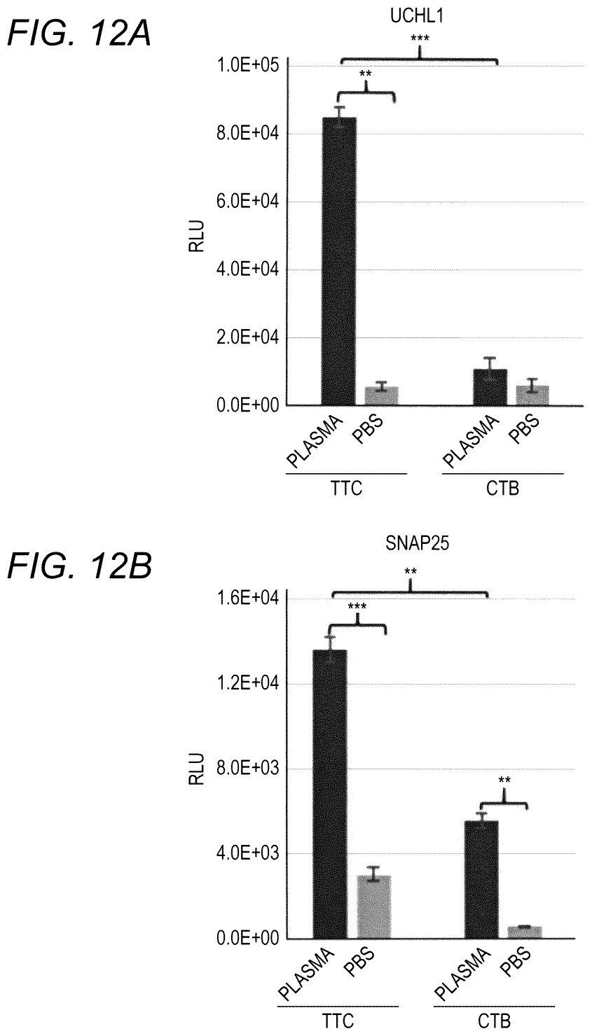

The measurement results are shown in FIGS. 8A to 10C. In the figure, “*” represents p≤0.05, “**” represents p≤0.01, “GRIA1” represents an anti-GRIA1 antibody, “SYT1” represents an anti-SYT1 antibody, “UCHL1” represents an anti-UCHL1 antibody, and “control” represents an isotype control. As shown in FIGS. 8A, 9A, and 10A, the signals of GRIA1, SYT1, and UCHL1 significantly increased in the case where plasma was added to the microplate as compared with the case where plasma was not added. As shown in FIGS. 8B, 9B, and 10B, the signals of GRIA1, SYT1, and UCHL1 significantly increased in the case of using the plate on which TTC was immobilized as compared with the case of using the plate on which TTC was not immobilized. As shown in FIGS. 8C, 9C, and 10C, the signals significantly increased in the case of using the anti-GRIA1 antibody, the anti-SYT1 antibody, and the anti-UCHL1 antibody as compared with the case of using the isotype control. From these measurement results, it was shown that the method of capturing EVs in plasma by TTC and detecting the captured EVs with the anti-GRIA1 antibody, the anti-SYT1 antibody, and the anti-UCHL1 antibody is a highly specific measurement method for NDEVs. Therefore, it was shown that NDEVs in a biological sample can be specifically measured by a solid-phase ligand binding assay using TTC immobilized on a solid phase and an antibody against a neuron-specific marker.

### Example 5: Comparison with Solid-Phase Ligand Binding Assay Using Cholera Toxin B

As described above, GM1 ganglioside to which CTB binds is known to be a molecule highly expressed in neurons of the brain. In this example, it was examined whether or not extracellular vesicles in a biological sample can be detected by a solid-phase ligand binding assay using CTB instead of TTC as a capture body of extracellular vesicle. For comparison, a solid-phase ligand binding assay using TTC was also performed.

### (1) Immobilization of CTB on Solid Phase (Microplate)

As CTB, a cholera toxin subunit B-biotin conjugate (Sigma-Aldrich) was used. A solution of CTB solubilized in PBS (2 μg/mL) was added to each well of a Maxisorp (trademark) 96-well plate (Thermo Fisher Scientific), and the mixture was incubated at 4° C. overnight. The solution was removed from each well, and then the plate was washed three times with a HISCL washing solution.

### (2) Solid-Phase Ligand Binding Assay

A solid-phase ligand binding assay was performed in the same manner as in Example 3 using CTB immobilized on a 96-well plate and a primary antibody and a secondary antibody described later. As primary antibodies, an antibody anti-CD81 mouse monoclonal antibody (Thermo Fisher Scientific), and an anti-SNAP25 rabbit polyclonal antibody (Proteintech), an anti-GRIA2 rabbit polyclonal antibody (Proteintech), and an anti-VILIP1 rabbit polyclonal antibody (Proteintech) were used. The same anti-CD9 antibody as in Example 3, and the same anti-SYT1 antibody and anti-UCHL1 antibody as in Example 4 were also used. As secondary antibodies, the same HRP-labeled rabbit anti-mouse IgG Fc antibody as in Example 3 and the same HRP-labeled goat anti-rabbit IgG antibody as in Example 4 were used. For comparison, an experiment using a plate on which TTC was immobilized instead of CTB and an experiment in which PBS was added instead of plasma were also performed.

### (3) Results

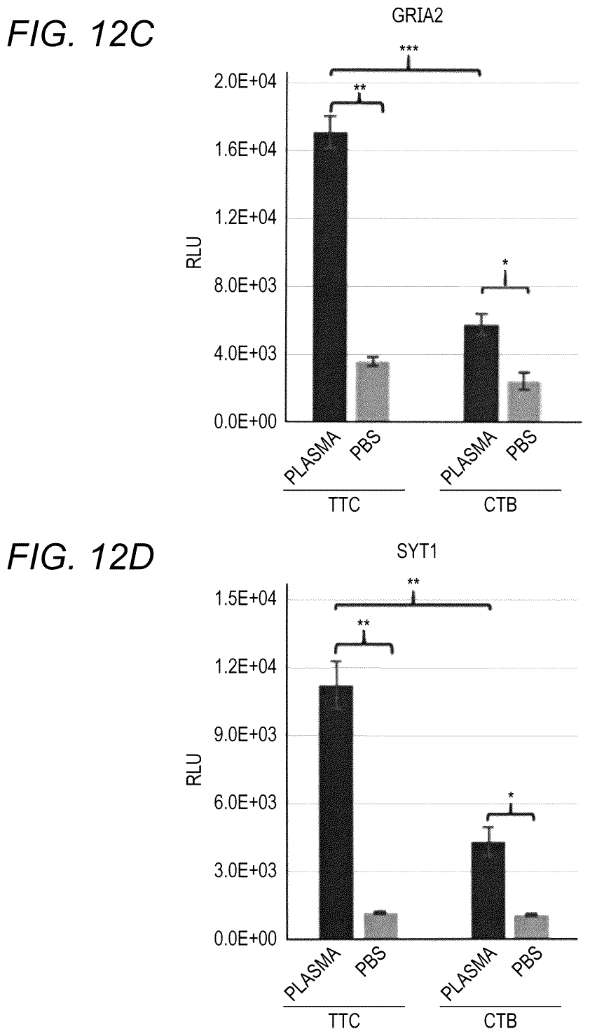

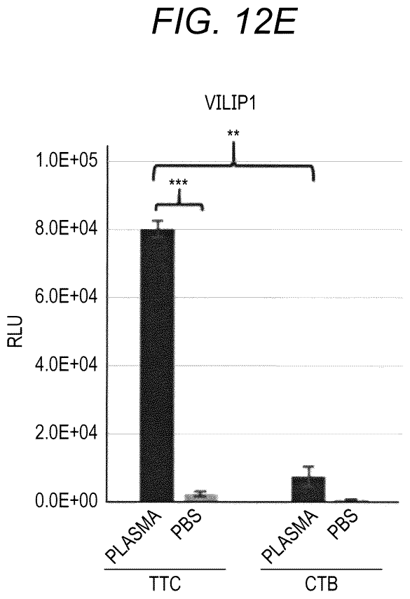

The measurement results are shown in FIGS. 11A to 12E. In the figure, “*” represents p≤0.05, “**” represents p≤0.01, and “***” represents p≤0.001. As shown in FIG. 11A, even when either CTB or TTC was used as the capture body, the signal of CD9 showed a high level. CD81 is an exosome marker protein as with CD9, but as shown in FIG. 11B, the level of the signal of CD81 was about 2 times higher when using TTC than when using CTB. The possibility that EV captured by TTC and EV captured by CTB are derived from different populations was suggested. As shown in FIGS. 12A to 12E, the signals of UCHL1, SNAP25, GRIA2, SYT1 and VILIP1, which are neuron-specific markers, all showed significantly higher levels when using TTC than when using CTB. Table 1 shows, for each marker, the ratio of the signal when using TTC to the signal when using CTB (TTC/CTB).

TABLE 1

| Neuron-specific marker | Signal ratio (TTC/CTB) |
|---|---|
| UCHL1 | 7.6 |
| SNAP25 | 2.5 |
| GRIA2 | 2.9 |
| SYT1 | 2.6 |
| VILIP1 | 10.6 |

As can be seen from FIGS. 12A to 12E and Table 1, it was shown that NDEV can be specifically captured by using TTC instead of CTB as a capture body of EV. That is, it was shown that the method of capturing EVs in plasma by TTC and detecting the captured EVs with an antibody against a neuron-specific marker is a highly specific measurement method for NDEVs.

### Example 6: Evaluation of Clinical Specimen

NDEVs in plasma obtained from subjects with dementia and healthy subjects were measured, and the relationship between the measurement results and cognitive function was examined.

### (1) Biological Samples

Plasma obtained from five healthy subjects (hereinafter, referred to as “HC”), five subjects with mild cognitive impairment (hereinafter referred to as “MCI”), and five subjects with Alzheimer's dementia (hereinafter, referred to as “AD”) was used as biological samples. Table 2 shows information on each subject.

TABLE 2

| | | MMSE | Aβ42 Concentration |
|---|---|---|---|
| Subject | Age | Score | in CSF (pg/mL) |
| AD | 80 | 20 | 177.3899 |
| AD | 63 | 15 | 109.3688 |
| AD | 76 | 20 | 303.9406 |
| AD | 65 | 20 | 283.2714 |
| AD | 56 | 14 | N/A |
| MCI | 67 | 27 | 189.0780 |
| MCI | 75 | 25 | 256.8827 |
| MCI | 55 | 25 | 289.7966 |
| MCI | 79 | 28 | 445.5365 |
| MCI | 63 | 23 | 217.6176 |
| HC | 76 | 30 | 635.6984 |
| HC | 71 | 30 | 884.6988 |
| HC | 56 | 30 | N/A |
| HC | 73 | 30 | N/A |
| HC | 63 | 30 | N/A |

### (2) Solid-Phase Ligand Binding Assay

NDEVs in plasma of each subject above were measured by a solid-phase ligand binding assay using a 96-well plate on which TTC was immobilized in the same manner as in Example 3. As primary antibodies, the same anti-SYT1 antibody as in Example 4 and the same anti-VILIP1 antibody as in Example 5 were used. As a secondary antibody, the same HRP-labeled goat anti-rabbit IgG antibody as in Example 4 was used.

### (3) Results

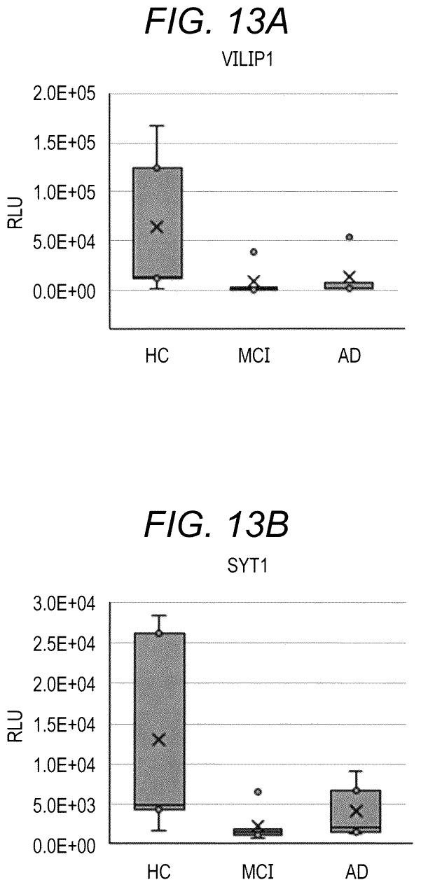

The measurement results are shown in FIGS. 13A and 13B. In the figure, “x” represents an average value of the signal levels. As shown in FIGS. 13A and 13B, the signals of VILIP1 and SYT1 were at extremely low levels in MCI and AD. On the other hand, in HC, the signal level greatly varied depending on the subject, but the average value thereof was sufficiently higher than those of MCI and AD. Here, in a large number of studies, it has been reported that protein molecules of VILIP1 and SYT1 are significantly reduced in the brain of Alzheimer's dementia patient. The measurement results of this example were consistent with the results already reported for the postmortem brains. From these results, it was suggested that the measured values of NDEVs obtained by a solid-phase ligand binding assay using TTC immobilized on a solid phase and an antibody against a neuron-specific marker might reflect the cognitive function of the subject, which is a type of symptom due to neurodegeneration.

What is claimed is:

1. A method for measuring neuron-derived extracellular vesicles in a biological sample in vitro, the method comprising:
forming a complex comprising a capture body comprising a tetanus toxin C-terminal fragment, the extracellular vesicle, a detector that specifically binds to a target molecule of the extracellular vesicle, and a labeling substance on a solid phase; and
measuring extracellular vesicles having the target molecule based on a signal generated by the labeling substance comprised in the complex.

2. The method according to claim 1, wherein the target molecule is at least one selected from a group consisting of VILIP1, SYT1, UCHL1, SNAP25, GRIA1, GRIA2, amyloid β, phosphorylated tau, CD9, CD63, and CD81.

3. A method for acquiring information on neurodegeneration of a subject, the method comprising:
forming a complex comprising a capture body comprising a tetanus toxin C-terminal fragment, a neuron-derived extracellular vesicle, a detector that specifically binds to a target molecule of the extracellular vesicle, and a labeling substance on a solid phase; and
detecting a signal generated by the labeling substance comprised in the complex,
wherein the target molecule is at least one selected from a group consisting of VILIP1, SYT1, UCHL1, SNAP25, GRIA1, GRIA2, amyloid β, and phosphorylated tau,
the forming and the detecting are performed in vitro, and
the measured value obtained in the detecting is an indicator of neurodegeneration of the subject.

4. The method according to claim 1, further comprising removing unreacted free components not forming the complex between the forming and the detecting.

5. The method according to claim 1, wherein
the forming comprises:
mixing the capture body immobilized on the solid phase with the biological sample; and
mixing a mixture of the capture body and the biological sample with the detector.

6. A method for isolating neuron-derived extracellular vesicles in a biological sample in vitro, the method comprising:
binding a capture body comprising a tetanus toxin C-terminal fragment to the extracellular vesicle; and
removing unreacted free components not bound to the capture body.

7. The method according to claim 6, wherein in the binding, the capture body and the extracellular vesicle are bound on the solid phase by mixing the solid phase on which the capture body is immobilized with the biological sample.

8. The method according to claim 1, wherein the biological sample is a blood sample, cerebrospinal fluid, urine, saliva, tear, lymph fluid, bronchoalveolar lavage fluid, or ascites.

9. The method according to claim 8, wherein the blood sample is whole blood, plasma, or serum.

10. The method according to claim 1, wherein the solid phase is a particle or a microplate.

11. A reagent kit for use in the method according to claim 1, comprising a capture body comprising a tetanus toxin C-terminal fragment.

12. The reagent kit according to claim 11, further comprising a detector that specifically binds to a target molecule of the extracellular vesicle.


SEQUENCE LISTING (5 sequences)

SEQ ID NO: 1 - protein, 451 aa, Clostridium tetani
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 2 - protein, 372 aa, Clostridium tetani
NDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 3 - protein, 450 aa, synthetic construct
  REGION 1..450: note = mutant of TTC
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEWTND

SEQ ID NO: 4 - protein, 451 aa, synthetic construct
  REGION 1..451: note = mutant of TTC
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTAEGWTND

SEQ ID NO: 5 - protein, 206 aa, Clostridium tetani
SITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND